(19) United States
(12) Patent Application Publication
(10) Pub. No.: US 2003/0228269 A1
(43) Pub. Date: Dec. 11, 2003

(54) Use of stilbene derivatives for dandruff treatment

(76) Inventors: Mario DeRosa, Naples (IT); Mose Rossi, Naples (IT)

Correspondence Address: Karen A Lowney, Estee Lauder Co, 125 Pinelawn Road, Melville, NY, 11747, US

(21) Appl. No.: 10/296,726

(22) PCT Filed: May 29, 2001
(86) PCT No.: PCT/EP01/06102

(30) Foreign Application Priority Data
Jun. 2, 2000 (IT) NA2000A000036

(51) Int. Cl. A61K 31/075; A61K 31/704; A61K 7/06; A61K 31/05

(52) U.S. Cl. 424/70.1; 514/25; 514/720; 514/733

(57) ABSTRACT

The present invention relates to the use of resveratrol and its derivatives as active principles in antidandruff formulations.

### FIELD OF THE INVENTION

The present invention relates to the use of resveratrol and its derivatives as active principles in antidandruff formulations.

### BACKGROUND OF THE INVENTION

There is an increasing need for innovative strategies in the dandruff treatments. Dandruff is due to the necrosis of epidermal cells and, in its pathological form, is an inflammatory disease which appears as dry or greasy diffuse scaling of the scalp with variable itching. The main cause of dandruff, which appears as flakes of the skin shed from the scalp in larger amounts than normal, is a reaction of cutis toward the yeast Pytirosporum ovale, whose abnormal growth is generally coupled with the presence of dandruff.

In addition to be non-aesthetic, dandruff may inhibit hair growth and generate hairs loss and infections of the scalp as it becomes the nutrient medium for the growth and proliferation of a number of microorganisms.

Resveratrol (3,4,5-trihydroxystilbene) is a phenolic stilbene and the parent natural glycosides are called polydatin or piceid. The trans isomer occurs in a narrow range of spermatophytes, including principally vines, peanuts and pine trees. Resveratrol is classified as an antifungal phytoalexin, conferring disease resistance in the plant kingdom. Its synthesis in plants is induced by stress, including infection or UV-irradiation. In vivo and in vitro experiments have shown that resveratrol possesses many biological properties. Recently, high concentrations of resveratrol have been found in the rhizomes of the plant Poligonum cuspidatum, so that this compound is now easily available for use in the pharmaceutical, cosmetic and nutritional fields. Resveratrol exerts potent anti-oxidant action, vasorelaxing effect and inhibition of pro-atherogenic eicosanoids by human platelets and neutrophils, activities that synergistically favor cardiovascular protection (The Lancet, 341:1103-1104, 1993; Neuroreport, 8:1499-1502, 1997; Chim Pharm Bull, 12:128-129, 1996; Chem Pharm Bull, 30:1766-70, 1982; Clin Chim Acta, 235:207-219,1995; Int J Tiss Reac, XVII:1-3,1995; Thrombosis and Gaemostasis, 76:818-819, 1996; Gen. Pharm., 27: 363-366, 1997). Resveratrol exerts anti-inflammatory action due to down-regulation of prostaglandin and prostacyclin synthesis and to the inhibition of cyclooxygenase and hydroperoxidase activities (Arch Pharm Res, 13:132-135, 1990; Science, 267:1782-1788, 1995; Bioch. Biophys. Acta, 834: 275-278, 1995). Resveratrol has also been shown to act as an antimutagen, by inhibiting the cellular events associated with tumor initiation, promotion and progression (Chem Pharm Bull, 30:1766-70, 1982; Science, 267:1782-1788, 1995; Am J Enol Vitic, 46:159-165, 1996; Science, 275:218-220, 1997; Cancer Res, 54:5848-5855, 1994; Anticancer Res, 14:1775-1778, 1995; Anal Biochem, 169:328-336, 1988; Proc Natl Acad Sci USA, 91:3147-3150, 1994; Proc Natl Acad Sci USA, 72:1848-1851, 1975; Carcinogenesis, 8:541-545, 1987).

A series of recent patents WO9959561; WO9958119; EP0773020; FR2766176; WO9904747 claim the use of resveratrol in the pharmaceutical and cosmetic fields.

However, none of the well-known above described properties of resveratrol could envisage the use of resveratrol in the treatment of dandruff.

The present invention relates to compositions for the topical application, containing resveratrol or its derivatives, of formula (I)

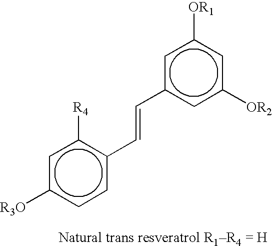

wherein:

R₁, R₂, R₃ are H; C₁-C₃₆ alkyl groups, optionally substituted by OH groups and optionally comprising one or more double bonds; C₂-C₃₆ acyl groups, optionally substituted by OH groups and optionally comprising one or more double bonds; a —(CH₂—CH₂—O)ₙ—H group where n is an integer from 1 to 30; or a glycosidic residue; and R₄ is H or OH.

Preferred resveratrol derivatives according to the invention are the ethers, esters, hydroxylated and glycosylated derivatives.

The compositions of the invention may be formulated, for example, in the form of lotions, creams, shampoos and hair conditioners, optionally in combination with other active principles.

Nor local neither systemic side effects have been observed during and after the application.

It has now surprisingly been found that resveratrol and its ethers, esters, hydroxylated and glycosylated derivatives can be effectively used in the treatment of dandruff.

It has also been found that a significant improvement of resveratrol efficacy in the treatment of dandruff can be obtained when using acid solutions containing the active principle. Therefore, preferred compositions of the invention contain resveratrol in acidic solution.

Resveratrol as an anti-dandruff agent offers the following advantages compared with the conventional antidandruff agents of the prior art:

a) it is a natural compound present in many food stuff and it is not toxic in the topic use, contrary to the most common antidandruff agents;

b) it is a stable natural compound which can be extracted in sufficient quantity, at a reasonable price, from the roots of the plant Polygonum cuspidatum;

c) its potent anti-oxidant action prevents the peroxydation of lipids of the cutis, a process which enhances the degeneration of the scalp microbial flora;

d) it has anti-aging action on the scalp and hairs due to the coupled effect of anti-radical action and vaso-relaxing action, which improves blood circulation in tissues and hair bulbs;

e) thanks to its regulatory effects on cellular growth, it acts against the proliferation phenomena which are at the basis of dandruff formation;

f) its anti-inflammatory action reduces the irritation phenomena associated with dandruff formation, reducing also the itching;

g) it is easily soluble in the components usually utilised in the formulation of cosmetic preparations, allowing to reach the desired concentration;

h) the lipophilic derivatives of resveratrol, ethers and esters with alcohols and long chain carboxylic acids, and hydrophilic derivatives, ethoxylated and glycosilated, allow the preparation of cosmetic formulations with optimal resistance to water or hydro-soluble, respectively.

The following examples further illustrate the invention.

### EXAMPLES

### Example 1

Lotion Containing 1% w/w Resveratrol

The lotion was prepared dissolving 1 g of pure resveratrol in 99 g of 1,4-butylenglycol:ethanol:water (3:3:4 by weight).

### Example 2

Lotion Containing 0.5% w/w Resveratrol at Acidic pH

The lotion was prepared dissolving 0.5 g of pure resveratrol in 59.5 g of butylene glycol:ethanol (1:1 w/w). The obtained solution was diluted with 40 g of 10 mM citrate buffer pH 4.0.

### Example 3

Experimental Approach for the Evaluation of the Antidandruff Action of the Lotion According to the Example 1 on Humans

Patient selection—The patients, between 18 and 60 years old, of both sex, with clinical findings consistent with dandruff capitis problems were approached regarding participation in a prospective, random, non blinded clinical trial. Informed consent was obtained for all patients who agreed to participate. At time of entry into the study, a clinical examination of the patient's scalp was performed and the findings were documented in the patient's medical chart. A dermatophyte culture specimen was obtained by vigorously brushing the affected area of the patient's scalp with a sterile toothbrush. The toothbrush bristles were then inoculated onto a Sabouraud's glucose agar plate, which was sent to the mycology laboratory for incubatiori. This diagnostic technique is similar to inoculation of the medium with the patient's hairbrush and is easy to perform.

Assessments—On the basis of predetermined random assignment, each patient received either 1% resveratrol lotion according to example 1, or a bland, non-medicated mixture and instructed to massage with the given product once a day for one week. Patients returned after 1 week to the clinical observation, and they were re-examined and recultured. At this time the physical examination findings were again documented in the patient's chart and the use of lotion product was reviewed with the patient and his or her family.

Results—Of 22 patients approached regarding study participation, 18 were enrolled in the study. 4 patients who had a positive dermatophyte culture were included in the study. There were no significant differences in gender assignment among the two treatment groups. At the 1-week visit, none of 9 patients who used the control lotion had reduction of dandruff. Two of 9 patients who used 1% resveratrol lotion had a significant decrease in dandruff at four day-treatment. As the study progressed, conversion to negative dandruff presence occurred at varying intervals in all treated patients.

### Example 4

Experimental Approach for the Evaluation for the Antidandruff Action on Humans of the Resveratrol Lotion Prepared According to the Example 2.

Patient selection—All patients were selected according to the example 3.

Assessments—On the basis of predetermined random assignment, each patient received either of the acidic 0.5% resveratrol lotion according to example 2, or a bland, non-medicated mixture and instructed to massage with the given product once a day for one week. Patients returned after 1 week to the clinical observation, and they were re-examined and recultured. At this time the physical examination findings were again documented in the patient's chart and use of lotion product was reviewed with the patient and his or her family.

Results—Of 30 patients approached regarding study participation, 23 were enrolled in the study. 5 patients who had a positive dermatophyte culture were included in the study. There were no significant differences in gender assignment among the two treatment groups. At the 1-week visit, none of 11 patients who used the control lotion had reduction of dandruff. Five of 12 patients who used 1% resveratrol lotion had a significant decrease in dandruff at four day-treatment. As the study progressed, conversion to negative dandruff presence occurred at varying intervals in all treated patients.

1. Use in the cosmetic treatment of dandruff of resveratrol and its ethers, esters and hydroxylated, ethoxylated and glycosylated derivatives, of formula (I) 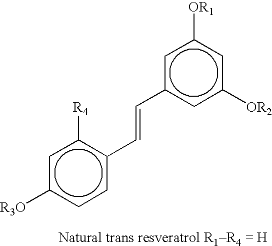
wherein:
R₁, R₂, R₃ are C₁-C₃₆ alkyl groups, optionally substituted by OH groups and optionally comprising one or more double bonds; C₁-C₃₆ acyl groups, optionally substituted by OH groups and optionally comprising one or more double bonds; a —(CH₂—CH₂—O)ₙ—H group where n is an integer from 1 to 30; or a glycosidic residue; and R₄ is H or OH.

2. Use as claimed in claim 1, wherein the resveratrol ether derivatives have formula (I), wherein at least one of R₁, R₂, R₃ is a C₁-C₃₆ alkyl group, optionally substituted by OH groups and optionally comprising one or more double bonds, and the others can be H; and R₄ is H.

3. Use as claimed in claim 1, wherein the resveratrol ester derivatives have formula (I), wherein at least one of R₁, R₂, R₃ is a C₁-C₃₆ acyl group, optionally substituted by OH groups and optionally comprising one or more double bonds, and the others can be H; and R₄ is OH.

4. Use as claimed in claim 1, wherein the resveratrol ethoxylated derivatives have formula (I), wherein at least one of R₁, R₂, R₃ is a —(CH₂—CH₂—O)ₙ—H group where n is an integer from 1 to 30, and the others can be H; and R₄ is H.

5. Use as claimed in claim 1, wherein resveratrol glycosylated derivatives have formula (I), wherein at least one of R₁, R₂, R₃ is a glycosidic residue, and the others can be H; and R₄ is H.

6. Antidandruff preparations comprising resveratrol derivatives according to the claims 1-5 and a cosmetic acceptable carrier selected from the group consisting of a solution, an oil, a cream, a lotion, a gel and a powder and auxiliary agents selected from the group consisting of thickeners, emulsifiers, preservatives and fragrances, and having an acid pH between 3.5 and 5.0.

7. Antidandruff preparations according to claim 6 containing 0.01 to 30% w/w resveratrol or derivatives thereof, preferably 0.1 to 5% w/w.

8. Antidandruff preparations, according to the claims 6 or 7 containing resveratrol derivatives, according to the claims 1-5, in association with coal tar, pyrition and its derivatives, undecilenic acid and its derivatives and anti-fungine and anti-inflammatory compounds.
